(19) United States
(12) Patent Application Publication
(10) Pub. No.: US 2023/0293424 A1
(43) Pub. Date: Sep. 21, 2023

(54) SHAMPOO VETERINARY COMPOSITIONS

(71) Applicant: Ceva Santé Animale, Liboume (FR)

(72) Inventors: Frédéric Lucas, Libourne (FR); Emeline Commeurec, Libourne (FR); Nadége Maubert, Libourne (FR)

(21) Appl. No.: 17/780,749

(22) PCT Filed: Nov. 27, 2020
(86) PCT No.: PCT/EP2020/083607

§ 371 Date: Nov. 16, 2022

(30) Foreign Application Priority Data
Nov. 28, 2019 (EP) 19212292.7

(51) Int. Cl. A61K 8/9783; A61K 8/46; A61K 8/60; A61Q 5/02

(52) U.S. Cl. CPC A61K 8/9783; A61K 8/60; A61K 8/466; A61Q 5/02

(57) ABSTRACT

The present invention relates to the veterinary field and, more particularly, to shampoo compositions comprising an active ingredient obtained from Ophiopogon japonicus and an amphoteric surfactant, for non-human mammals. The invention also relates to a method for washing the coat of non-human mammals by applying said shampoo compositions to the coat and/or the skin.

### OBJECT OF THE INVENTION

The present invention relates to the veterinary field, and more particularly to shampoo compositions comprising an active ingredient obtained from Ophiopogon japonicus, and an amphoteric surfactant, intended for non-human mammals. It also relates to a method for washing the coat of non-human mammals by applying said shampoo compositions to the coat and/or the skin.

### TECHNICAL BACKGROUND OF THE INVENTION

The active ingredient obtained from Ophiopogon japonicus, and the hydrating effect thereof or the effect thereof for reinforcing the barrier function of human skin has been described in the patent EP2271311. The use of said active ingredient for treating atopic dermatitis in animals and humans has been described in the patent application WO2017/121965.

However, no mention is made of a shampoo comprising said active ingredient which is suitable for non-human mammals, in particular dogs and cats. The skin and the coat of non-human mammals protect said mammals from external aggressions. Their upkeep is therefore very important for the well-being, and indeed the health, of non-human mammals. The skin and the hairs of domestic animals such as dogs and cats are covered by a layer of fatty acids and waxes which contribute to the barrier function, thus having a role of protecting the skin from external aggressions, and which give the silky appearance of the coat.

A veterinary shampoo must therefore be effective in terms of washing power for the skin and the coat, without causing irritation or any other skin disorder. It must therefore also preserve or maintain the barrier function and/or the silky appearance of the coat. The skin must in particular keep its protective effect with respect to external aggressions.

Furthermore, bacteria exist which may be pathogenic or become pathogenic if they are too numerous, such as the staphylococcaceae, for dogs and/or cats, and which require particular treatments in view of their reduction or elimination. These staphylococcaceae are represented primarily by S. pseudintermedius.

There is therefore a need for shampoo compositions for non-human mammals, and in particular for dogs and cats, which are both effective in terms of washing, and which make it possible to preserve, or indeed improve, the barrier function of the skin and of the coat and/or the silky appearance of the coat, and which make it possible to preserve the bacterial flora which is beneficial to the mammals, while limiting, or indeed preventing, the colonization of the skin by pathogenic bacteria.

### SUMMARY OF THE INVENTION

In this context, the applicant is proposing a new shampoo composition comprising an active ingredient obtained from Ophiopogon japonicus, and at least one amphoteric surfactant selected from the betaines.

The present invention thus relates to a veterinary composition comprising an active ingredient obtained from Ophiopogon japonicus, and at least one amphoteric surfactant selected from the betaines, which is intended to be used as a shampoo for a non-human mammal, in particular a dog or a cat.

A further object of the invention thus relates to a method for cleaning and/or washing the skin and/or the coat of a non-human mammal, and in particular a dog or a cat, by applying the composition of the present invention to the skin and/or the coat of said mammal.

A further object of the invention relates to the use of a composition as defined in the present application, or a method using said composition for reducing and/or preventing the presence of pathogenic bacteria and/or the formation and/or the adhesion of pathogenic bacteria biofilms in the case of a non-human mammal, such as a dog or cat.

### DETAILED DESCRIPTION OF THE INVENTION

The invention as described in the present application relates to a shampoo veterinary composition comprising an active ingredient obtained from Ophiopogon japonicus, and at least one amphoteric surfactant selected from the betaines.

Ophiopogon japonicus is a perennial, low and abundant herbaceous species having rhizomes, from the family of the Liliaceae. It is cultivated as an ornamental covering plant having tuberous rhizomes, and has a very large number of uses in traditional Chinese medicine.

The species is originally from Japan and Korea, and is also cultivated in Vietnam and in China, in particular in Sichuan, Zhejiang and Hubei provinces. It is also referred to as “Muguet du Japon” in France, and “Mondo grass,” “Fountain plant,” “Monkey grass” or “Dwarf lilyturf” in English. The tuberous root is of a length of a few centimeters; it is light yellow to yellow-brown in color on the outside, having longitudinal wrinkles. Its smell is slight, and its taste slightly sweet and mucilaginous.

In traditional Chinese medicine, the root is known to “feed the Lungs and the Yin, to feed the Stomach and produce Fluids, to remove the Heat from the heart, and to soothe the Spirit.” The main therapeutic indications relate to dry coughs, sore throats, insomnia, irritability, constipation and diphtheria, according to the 16th edition of the Japanese Pharmacopeia.

The roots of Ophiopogon japonicus originate from strains cultivated in China and Vietnam. Extracts of Ophiopogon japonicus are known in cosmetics for the skin, in particular in the patent EP2271311, as a moisturizer by action on the rate of NMF (Natural Moisturizing Factors) and on the formation of tight junctions of the cutaneous layers. The application WO 2017/121965 also describes that extracts of Ophiopogon japonicus can be used for treating atopic dermatitis.

Within the meaning of the invention, “active agent” or “active ingredient” or “extract” means at least one molecule, preferably a set of molecules, having an effect on the cells of the skin.

Within the meaning of the invention, “active agent obtained from Ophiopogon japonicus” means any molecule or mixture of molecules obtained from Ophiopogon japonicus. They may be native molecules of the plant, or molecules obtained by any kind of transformation of native molecules of the plant, for example by hydrolysis. The active agent according to the invention is preferably a hydrolysate.

“Hydrolysate” means any extract originating from Ophiopogon japonicus which is obtained by means of a method comprising at least one step of enzymatic or chemical hydrolysis of Ophiopogon japonicus, preferably at least one step of enzymatic hydrolysis. “Ophiopogon japonicus” means all or part of the plant. It may be the entire plant, or a part of the plant. It is preferably tubers of Ophiopogon japonicus.

“Oligosaccharides” means oligomers formed of a number of monosaccharides by glycosidic bonding, the number of units of monosaccharides being less than 25 units. “Polysaccharides” means polymers formed of a plurality of simple sugars which are interconnected by osidic bonding, the number of units of monosaccharides being greater than 25 units.

The active agent obtained from Ophiopogon japonicus, used in the composition according to the invention, is preferably an active agent obtained from Ophiopogon japonicus comprising sugars. Even more preferably, it comprises fructosans, and more particularly it comprises at least 57% fructosans by weight with respect to the total weight of sugars of the active agent, even more preferably at least 80%. The fructosans are polysaccharides made up of fructose and glucose. The sugars containing the active agent are preferably made up of 45 to 80% fructose, 20 to 50% glucose, and 0 to 5% galactose. Said sugars may be in the form of monomers, oligomers and polymers. Predominantly, the sugars contained in the active agent are oligo- and polysaccharides of molecular weights of less than 400 kDa, in the form of fructosans. Thus, the active agent according to the invention preferably comprises oligo- and polysaccharides of molecular weights of less than 400 kDa, in the form of fructosans, representing at least 57 wt.% of the sugars present in the active agent. According to a particularly suitable variant, the active agent is obtained from tubers of Ophiopogon japonicus.

According to an embodiment, the active agent used for preparing the composition according to the invention is present in the form of a powder, in particular a clear-colored powder, and has at least one of the following features, preferably all:
an amount of dry matter of between 900 and 1000 mg/g, or
a sugar content of between 500 and 800 mg/g, i.e. at least 50% sugars by weight with respect to the weight of dry matter.

The amount of dry matter may be measured by passing a sample into a sterilizer at 105° C. until a constant weight is achieved.

The overall content of sugars may be determined by the DUBOIS method, on a range of fructose (Dubois M. et al., Analytical Chemistry, 28, 3, 350-356, 1956).

The characterization of the molar mass of the carbohydrates present in the active agent of the present invention may be achieved by an HPLC method and by metering of simple sugars by ionic liquid chromatography.

The molar masses of the carbohydrates are evaluated by comparing the retention times of the peaks detected in the samples of the active agent with the retention times of standards injected previously.

The operating conditions are preferably as follows:
Apparatus: Agilent 1100 Series HPLC
Columns: PL aquagel-OH C60, C40, C30 columns having a pre-column of the same characteristics
Elution mode: isocratic
Mobile phase: Buffer NaN030.3M + NaH2P04-2H20 0.01 M pH 7
Detection wavelengths: UV 254 nm and 280 nm

The active agent according to the invention is preferably an active agent obtained in aqueous medium from tubers of Ophiopogon japonicus. “Obtained in aqueous medium” means a medium containing mainly water, or a basic or acidic aqueous medium. In particular, it is not an oil or an essential oil.

The active agent according to the invention is preferably a hydrolysate of Ophiopogon japonicus, preferably an enzymatic hydrolysate.

According to a particularly suitable variant, the active agent according to the invention is a hydrolysate of Ophiopogon japonicus, preferably an enzymatic hydrolysate of tubers of Ophiopogon japonicus.

In particular, the active agent which can be used according to the invention can be obtained by implementing the following steps:
solubilization of powder of Ophiopogon japonicus (preferably of tubers) in water at the rate of at least 50 g/l,
at least one enzymatic hydrolysis of sugars,
separation of the soluble and insoluble phases, for example by settling,
enzymatic inactivation by heat treatment of the soluble phase.

The enzymatic inactivation may be followed by one or more steps of filtration and/or concentration. The active agent may be obtained in liquid form or in the form of powder, by atomization or lyophilization. It is preferably atomized, in the presence of an atomization additive of the maltodextrin type, and used in powder form.

The parameters of the various steps have to be adjusted in order to obtain active agents having the features of the invention, in particular the presence of fructosans having a molecular weight of less than 400 kDa.

According to a preferred embodiment, the active agent is atomized using maltodextrin; it is thus present in powder form (before formulation into the composition according to the invention).

The composition according to the invention advantageously comprises at least 0.1 wt.% active agent obtained from Ophiopogon japonicus, according to the present invention, preferably between 0.1 and 1 wt.% (or between 0.1 and 0.8 wt.%) with respect to the total weight of the composition, and more particularly between 0.1 and 0.6 wt.% with respect to the total weight of the composition.

The amphoteric surfactants of the betaine type are preferably selected from the alkyl(C8-C20)betaines, the alkyl(C8-C20)amidoalkyl(CI-C8)betaines, the alkyl(C8-C20)amidoalkyl(C1-C8)sulfobetaines, the alkyl(C8-C20)amidoalkyl(C1-C6)hydroxysulfobetaines, the sulfobetaines and the hydroxysulfobetaines. By way of example, particularly the compounds classified under the designations coco betaine, lauryl betaine, cetyl betaine, coco/oleamidopropyl betaine cocamidopropyl betaine, palmitamidopropyl betaine, stearamidopropyl betaine, cocamidoethyl betaine, cocamidopropyl hydroxysultaine, oleamidopropyl hydroxysultaine coco hydroxysultaine, lauryl hydroxysultaine, coco sultaine, alone or in mixtures, can be cited.

In particular, said at least one amphoteric surfactant of the betaine type is selected from the alkyl(C8-C20)amidoalkyl(C1-C6)betaines, the alkyl(C8-C20)amidoalkyl(C1-C6)sulfobetaines (also referred to as alkyl(C8-C20)amidoalkyl(C1-C6)sultaines), the alkyl(C8-C20)amidoalkyl(C1-C6)hydroxysulfobetaines (also referred to as alkyl(C8-C20)amidoalkyl(C1-C6)hydroxysultaines). The amphoteric surfactant of the betaine type is preferably selected from the alkyl(C8-C20)amidoalkyl(C1-C6)hydroxysulfobetaines. It is more specifically cocamidopropyl hydroxysultaine (in particular marketed by the company Seppic under the trade name “Amonyl 675 SB”).

The composition according to the present invention advantageously comprises at least 3% amphoteric surfactants of the betaine type, preferably between 4 and 15% with respect to the total weight of the composition, and more particularly between 4 and 12% with respect to the total weight of the composition.

As used here, the term “shampoo” includes any composition intended to clean the hair or the skin of non-human mammals, and requires rinsing with water after application to the non-human mammal. Generally, shampoos have certain essential characteristics. Thus, a shampoo has moistening, foaming and detergent properties, in order to ensure satisfactory cleaning of the skin and of the coat.

In particular, the shampoo composition according to the invention contains, in addition to said at least one amphoteric surfactant selected from the betaines, one or more additional surfactant agents, preferably at least one additional anionic, cationic, amphoteric, nonionic surfactant agent, or a mixture thereof. According to a preferred embodiment, the shampoo composition according to the invention contains, in addition to said at least one amphoteric surfactant selected from the betaines, at least one additional anionic surfactant agent, at least one additional amphoteric surfactant agent (i.e. different from said at least one amphoteric surfactant selected from the betaines), at least one additional nonionic surfactant agent, or a mixture thereof.

From the anionic surfactants, the sulfonate or carboxylic anionic surfactants are preferably used.

According to the invention, the sulfonate anionic surfactants are anionic surfactants having at least one sulfonate function (—SO₃H or —SO₃⁻) and not having any cationic function.

The sulfonate anionic surfactants which can be used, alone or in mixtures, within the context of the present invention, are salts (in particular alkaline salts, in particular sodium, ammonium salts, amine salts, amino alcohol salts or magnesium salts), alkyl sulfonates, alkyl sulfosuccinates, alkyl ether sulfosuccinates, acyl isethionates, acyl methyl isethionates, methyl acyl taurates; the alkyl or acyl radical of all these different compounds preferably comprising from 8 to 24 carbon atoms, and the aryl radical preferably denoting a phenyl or benzyl group. The average number of ethylene oxide or propylene oxide groups may in particular be from 2 to 50, and more particularly from 2 to 10.

According to the invention, the carboxylic anionic surfactants are anionic surfactants having at least one carboxylic function (—COOH), optionally in the form of a salt (—COO⁻).

The anionic surfactants of the carboxylic type which are different from the surfactants preferably do not have any sulfate or sulfonate function, and can in particular be selected from the alkyl D-galactoside uronic acids and the salts thereof, the alkyl (C6-C24) ether carboxylic polyoxyalkylene acids, the alkyl (C6-C24) aryl ether carboxylic polyoxyalkylene acids and the salts thereof, the alkyl (C6-C24) amido ether carboxylic polyoxyalkylene acids and the salts thereof, in particular those comprising 2 to 50 alkylene, in particular ethylene, oxide groups such as the compounds proposed by the company KAO under the names AKYPO, the acyl (C6-C24) sarcosinates and the salts thereof, the acyl (C6-C24) lactylates and the salts thereof, and the acyl (C6-C24) glutamates. It is also possible to use the esters of carboxylic alkyl(C6-C24)polyglycosides such as the acetate alkyl glucosides, the citrate alkyl glucosides, and the tartrate alkyl polyglycosides. Such products are in particular marketed under the names Eucarol APG/EC and Eucarol APG/ET by the company Lamberti, and Plantapon LGC SORB by the company Cognis.

The salts are in particular selected from the alkaline salts, in particular of sodium, the ammonium salts, the amine salts, the aminoalcohol salts such as triethanolamine or monoethanolamine, and the magnesium salts.

The alkyl (C6-C24) ether carboxylic polyoxyalkylene acids and the salts thereof, the alkyl (C6-C24) amido ether carboxylic polyoxyalkylene acids and the salts thereof, in particular those comprising 2 to 15 alkylene oxide groups, the esters of alkyl (C6-C24) polyglycoside carboxylic esters and the salts thereof, and the acyl (C6-C24) glutamates and the salts thereof, and the mixtures thereof, are preferably used. The alkyl (C6-C24) ether carboxylic polyoxyalkylene acids and the salts thereof, the alkyl (C6-C24) amido ether carboxylic polyoxyalkylene acids, in particular those comprising 2 to 15 alkylene oxide groups, and the salts thereof, and the mixtures thereof, are more particularly used.

Of these anionic surfactants, sulfate-free surfactants are preferably used. According to a particular embodiment, the composition according to the invention comprises at least one anionic surfactant selected from the alkyl ether sulfosuccinates, the acyl methyl isethionates, the methyl acyl taurates. In particular, in the composition according to the invention disodium laureth sulfosuccinate, sodium lauroyl methyl isethionate, sodium methyl cocoyl taurate, or a mixture thereof, is used.

The anionic surfactants may be present in amounts of from 2 wt.% to 30 wt.%, preferably from 4 to 25 wt.%, particularly from 6 to 20 wt.%, more particularly from 8 to 15 wt.%, at best from 10 to 14 wt.%, with respect to the total weight of the composition. More specifically, the composition comprises disodium laureth sulfosuccinate, in particular marketed by the company Evonik under the trade name Rewopol SB 30 B. The amount of disodium laureth sulfosuccinate may be between 1 and 5 wt.%, preferably between 2 and 4 wt.%, with respect to the total weight of the composition.

More specifically, the composition comprises sodium lauroyl methyl isethionate, in particular marketed by the company Innospec under the trade name Iselux Flakes. The amount of sodium lauroyl methyl isethionate may be between 3 and 7 wt.%, preferably between 4 and 5 wt.%, with respect to the total weight of the composition.

More specifically, the composition comprises sodium methyl cocoyl taurate, in particular marketed by the company Innospec under the trade name Pureact WS Conc. The amount of sodium methyl cocoyl taurate may be between 3 and 7 wt.%, preferably between 4 and 5 wt.%, with respect to the total weight of the composition.

In particular, the composition comprises a mixture of disodium laureth sulfosuccinate, sodium lauroyl methyl isethionate, and sodium methyl cocoyl taurate, according to the quantities of each as indicated above.

The additional amphoteric surfactant agents other than those of the betaine type which may be used in the present invention can in particular (non-limiting list) be secondary or tertiary aliphatic amines, in which the aliphatic radical is a linear or branched chain comprising 8 to 22 carbon atoms and containing at least one anionic hydrosolubilizing group (for example carboxylate, sulfonate, sulfate, phosphate or phosphonate).

These compounds may be the following compounds: sodium cocoamphoacetate, sodium cocoyl glutamate, disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid (INCI names). The amphoteric surfactant agent(s) in addition to those of the betaine type are preferably selected from sodium cocoamphoacetate, sodium cocoyl glutamate, and a mixture thereof.

The additional amphoteric surfactant(s) other than those of the betaine type, and in particular sodium cocoamphoacetate and/or sodium cocoyl glutamate, are present in the composition according to the invention in amounts of from 0.1 to 10 wt.%, preferably from 1 to 5 wt.%, and more particularly from 2 to 4 wt.%, and at best from 2 to 3 wt.%, with respect to the total weight of the composition.

The nonionic surfactants which can be associated with these compounds and with these compositions include in particular the alkyl glucosides, the alkyl polyglucosides, the derivatives of castor oil, the polysorbates, the copra amides, the N-alkylamines, or the amine oxides.

According to a particular embodiment, the composition according to the invention comprises at least one nonionic surfactant selected from the alkyl (poly)glucosides, and in particular decyl glucoside, lauryl glucoside, cocoyl glucoside or capryl/caprylyl polyglucoside. The composition according to the invention preferably comprises decyl glucoside.

The nonionic surfactant(s) may be present in the composition according to the invention in amounts of from 0.1 to 15 wt.%, preferably approximately 1 to 10 wt.%, and more preferably approximately 3 to 8 wt.%, and at best 5 to 7 wt.%, with respect to the total weight of the composition.

The composition is preferably an aqueous composition. It may comprise water in amounts of from 30 to 95 wt.% with respect to the total weight of the composition, preferably from 40 to 90 wt.%, particularly from 50 to 85 wt.%.

The composition according to the invention may comprise other compounds such as modified or unmodified guar gum. More particularly, the composition according to the invention comprises cationic galactomannan gums (which are described more particularly in the patents US3589578 and US4031307). Guar gums comprising cationic trialkylammonium groups can be cited. For example, guar gums are used which are modified by a salt (for example a chloride) of 2,3-epoxypropyl trimethylammonium. Such products are marketed in particular under the name JAGUAR EXCEL by the company Solvay or SACI-CFPA. The modified or unmodified guar gums are advantageously present in the composition in amounts of between 0.01 and 0.5 wt.%, preferably between 0.05 and 0.15 wt.%, and at best between 0.08 and 0.1 wt.%, with respect to the total weight of the composition.

The composition according to the invention may comprise one or more chelating agents which may be selected in particular from EDTA (ethylenediaminetetraacetic acid) and the salts thereof such as disodium EDTA and dipotassium EDTA, the phosphate compounds such as sodium metaphosphate, sodium hexametaphosphate, tetrapotassium pyrophosphate, the phosphonic acids and the salts thereof such as the salts of ethylenediamine tetra(methylene phosphonic acid), and the mixtures thereof.

The shampoo compositions according to the invention may furthermore comprise agents which are beneficial for the skin or the coat. These ingredients are generally introduced into the composition in order to improve the touch qualities of the keratinic fiber of the coat or of the skin or indeed to promote the hydration thereof. The cationic polymers, the silicones and the emollients, such as the polyols, constitute, in this context, the most commonly used ingredients. It is also possible to use soothing agents, such as panthenol.

The composition can also comprise thickening or suspension agents, such as copolymers of acrylic or methacrylic acid, and of a chain of branched alkyl acrylate with an ether allyl of pentaerythritol or of saccharose. In particular the compound acrylate/C10-30 alkyl acrylate crosspolymer (INCI name) can be cited.

The composition according to the invention may further comprise other compounds, in particular selected from the saccharides, oligosaccharides, hydrolyzed or non-hydrolyzed polysaccharides, modified or unmodified, such as saccharide isomerate, amino acids, oligopeptides, peptides, hydrolyzed or non-hydrolyzed proteins, modified or unmodified, branched or unbranched fatty acids and alcohols, animal, vegetable or mineral oils or waxes, ceramides and pseudoceramides, hydroxylated organic acids, antioxidants, anti-free radical agents, cationic, anionic or nonionic polymers, soluble or dispersed, or a mixture of said compounds.

Of course, a person skilled in the art will be sure to select this or these possible additional compounds and/or their amounts in such a way that the advantageous properties intrinsically linked with the association according to the invention are not, or substantially not, altered by the intended addition(s).

Said additives are optionally present in the composition according to the invention in proportions which may be from 0.00001 to 30 wt.%, with respect to the total weight of the composition. The exact amount of each additive is easily determined by a person skilled in the art, depending on the type and function thereof.

Advantageously, the composition according to the invention may comprise one or more agents which are beneficial for the skin or the coat, and in particular hydrating agents, for example selected from panthenol, saccharide isomerate, and/or glycerin. The panthenol may be present in the composition in a content of from 0.1 to 3 wt.%, with respect to the total weight of the composition. The saccharide isomerate may be present in the composition in a content of from 0.001 to 1 wt.%, with respect to the total weight of the composition.

These shampoo compositions may be provided in the form of thicker or thinner liquids, and they are mainly suitable for washing, for care for keratinic materials, in particular the coat or the skin of the non-human mammal.

The invention also relates to a method for cleaning and/or washing the skin and/or the coat of a non-human mammal, and in particular a dog or a cat, by applying the composition of the present invention to the skin and/or the coat of said mammal.

The compositions according to the invention are implemented by simple application to the non-human mammal, optionally after having dampened all or some of the non-human mammal with water, and the foam generated, optionally by massage or friction using the hands, is subsequently removed, after an optional dwell time, by rinsing with water, it being possible for the operation to be repeated one or more times.

The invention also relates to a method for washing and conditioning the coat and/or the skin of the non-human mammal, comprising the application of a composition as defined above, then performing rinsing with water, after a possible dwell time. The skin and/or the coat are advantageously dampened prior to the application of the composition according to the invention.

The method according to the invention also makes it possible to be non-irritant, or indeed to reduce the irritation of the skin, which reduces the stress and provides an effect of well-being to the non-human mammal, in particular the dog or the cat. It also makes it possible to preserve, or indeed improve, the barrier function of the skin, in particular by virtue of reducing irritation, and/or the silky appearance of the coat.

The shampoo compositions according to the invention for non-human mammals, and in particular for dogs and cats, allow for effective washing, while preserving, or indeed improving, the barrier function of the skin and/or of the coat, and/or the silky appearance of the coat. The shampoo compositions according to the invention for non-human mammals, and in particular dogs and cats, also make it possible to reduce the presence of pathogenic bacteria or the development thereof, or to reduce and/or prevent the formation and/or the adhesion of pathogenic bacteria biofilms, the pathogenic bacteria being in particular Staphylococcus aureus and/or Staphylococcus pseudintermedius.

A further object of the invention relates to the use of a composition as defined above, or a method using said composition, for reducing and/or preventing the formation and/or the adhesion of pathogenic bacteria biofilms, or for reducing the presence, on the skin or the coat, of pathogenic bacteria, such as Staphylococcus aureus and/or Staphylococcus pseudintermedius, in a non-human mammal, in particular a dog or cat.

Thus, the use of a composition as defined above, or a method using said composition, for reducing and/or preventing the formation and/or the adhesion of pathogenic bacteria biofilms, or for reducing the presence of pathogenic bacteria, can be achieved by application of a composition as defined above, then by rinsing with water, following an optional dwell time. The skin and/or the coat are advantageously dampened prior to the application of the composition according to the invention.

According to a particular embodiment of the invention, the non-human mammal is selected from the felids, the canines, and/or the equids, and in particular dogs and cats.

The term “approximately” will be understood by a person skilled in the art, and can vary to a certain extent, depending on the context in which it is used. If some uses of this term are not clear to a person skilled in the art, depending on the context “approximately” means plus/minus 10%, preferably plus/minus 5%, of the value indicated.

The values indicated in the intervals specified in the text must be included, unless otherwise stated.

According to the invention, the term “comprises” or “comprise” (and other comparable terms, for example “containing” and “including”) is “open” and may be interpreted, in a general manner, such that the specific features mentioned, and all the optional, additional and non-specified features are included. According to specific embodiments, it may also be interpreted as an expression “essentially consisting in,” which includes the specific features and any optional, additional or non-specified feature which does not materially affect the fundamental and new feature or features of the claimed invention, or as an expression “consisting in,” where only the specific features are included, unless otherwise indicated.

According to the invention, the term “at least” means “one,” “two,” “three,” or more.

Other aspects and advantages of the invention will appear from reading the following examples, which should be considered as illustrative and non-limiting.

The percentages indicated are by weight, unless otherwise stated.

### Examples

The following compositions of tables 1 to 4 are prepared according to the conventional methods for preparing shampoo.

These compositions exhibit good washing properties. They make it possible to improve the barrier function, to restore the microbial balance, and in particular to reduce the presence of pathogenic bacteria in dogs or cats, and also to limit the adhesion or the formation of biofilm. The pathogenic bacteria are in particular Staphylococcus aureus and/or Staphylococcus pseudintermedius.

TABLE 1

| Compound Trade name (company) | Compound INCI name | % Compound in the composition |
|---|---|---|
| | AQUA | QS 100 |
| Carbopol Ultrez 20 (Lubrizol) | ACRYLATES/C10-30 ALKYL ACRYLATE CROSSPOLYMER | 0.3 |
| | SODIUM METHYL LAUROYL ISETHIONATE | 4 |
| Pureact WS Conc (Innospec) | SODIUM METHYL COCOYL TAURATE | 4 |
| Rewopol SB FA30 B (Evonik) | DISODIUM LAURETH SULFOSUCCINATE in aqueous solution at 41% MA | 7.5 |
| Rewoteric AM/C (Evonik) | SODIUM COCOAMPHOACETATE in aqueous solution at 50% MA | 5 |
| | DISODIUM ETHYLENEDIAMINE TETRAACETATE | 0.05 |
| | PRESERVATIVE | qs |
| | PANTHENOL | 2.0 |
| Jaguar Excel (Solvay) | GUAR HYDROXYPROPYLTRIMONIUM CHLORIDE at 95% MA with water | 0.1 |
| Amonyl 675 SB (Seppic) | COCAMIDOPROPYL HYDROXYSULTAINE in aqueous solution at 60% MA | 8 |
| Ophytrium (Ceva) | OPHIOPOGON JAPONICUS ROOT EXTRACT at 74% MA with maltodextrin | 0.75 |
| Pentavitin (DSM) | SACCHARIDE ISOMERATE in aqueous solution at 50% MA | 0.5 |

TABLE 2

| Compound Trade name (company) | Compound INCI name | % Compound in the composition |
|---|---|---|
| | AQUA | QS 100 |
| Carbopol Ultrez 20 (Lubrizol) | ACRYLATES/C10-30 ALKYL ACRYLATE CROSSPOLYMER | 0.3 |
| | SODIUM METHYL LAUROYL ISETHIONATE | 4 |
| Pureact WS Conc (Innospec) | SODIUM METHYL COCOYL TAURATE | 4 |
| Rewopol SB FA30 B (Evonik) | DISODIUM LAURETH SULFOSUCCINATE in aqueous solution at 41% MA | 7.5 |
| Rewoteric AM/C (Evonik) | SODIUM COCOAMPHOACETATE in aqueous solution at 50% MA | 5 |
| | PRESERVATIVE | qs |
| | PANTHENOL | 1.1 |
| Jaguar Excel (Solvay) | GUAR HYDROXYPROPYLTRIMONIUM CHLORIDE at 95% MA with water | 0.1 |
| Amonyl 675 SB (Seppic) | COCAMIDOPROPYL HYDROXYSULTAINE in aqueous solution at 60% MA | 8 |
| Ophytrium (Ceva) | OPHIOPOGON JAPONICUS ROOT EXTRACT at 74% MA with maltodextrin | 0.25 |
| Pentavitin (DSM) | SACCHARIDE ISOMERATE in aqueous solution at 50% MA | 0.5 |

TABLE 3

| | | |
|---|---|---|
| Compound Trade name (company) | Compound INCI name | % Compound in the composition |
| Water | AQUA | QS 100 |
| AMONYL 675 SB (Seppic) | COCAMIDOPROPYL HYDROXYSULTAINE in aqueous solution at 50% MA | 20 |
| | CHLORHEXIDINE DIGLUCONATE | 3 |
| Oramix NS10 (Seppic) | DECYL GLUCOSIDE in aqueous solution at 57% MA | 12 |
| | PANTHENOL | 0.75 |
| PENTAVITIN (DSM) | SACCHARIDE ISOMERATE in aqueous solution at 50% MA | 0.5 |
| | PEG-200 Glyceryl Stearate | 0.8 |
| Ophytrium (Ceva) | OPHIOPOGON JAPONICUS EXTRACT at 74% MA with maltodextrin | 0.5 |
| | PRESERVATIVE | qs |

TABLE 4

| Compound Trade name (company) | Compound INCI name | % Compound in the composition |
|---|---|---|
| Water | AQUA | QS 100 |
| Carbopol Ultrez 20 (Lubrizol) | ACRYLATES/C10-30 ALKYL ACRYLATE CROSSPOLYMER | 0.3 |
| | SODIUM METHYL LAUROYL ISETHIONATE | 5 |
| Pureact WS Conc (Innospec) | SODIUM METHYL COCOYL TAURATE | 5 |
| Rewopol SB FA30 B (Evonik) | DISODIUM LAURETH SULFOSUCCINATE in aqueous solution at 41% MA | 5 |
| Tego® Betain 810 (Evonik) | CAPRYLAMIDOPROPYL BETAINE / CAPRAMIDOPROPYL BETAINE in aqueous solution at 35.5% MA | 2.5 |
| | DISODIUM ETHYLENEDIAMINE TETRAACETATE | 0.05 |
| | PRESERVATIVE | QS |
| | PANTHENOL | 2.0 |
| Jaguar Excel (Solvay) | GUAR HYDROXYPROPYLTRIMONIUM CHLORIDE at 95% MA with water | 0.1 |
| Amonyl 675 SB (Seppic) | COCAMIDOPROPYL HYDROXYSULTAINE in aqueous solution at 60% MA | 12.5 |
| Ophytrium (Ceva) | OPHIOPOGON JAPONICUS ROOT EXTRACT at 74% MA with maltodextrin | 0.5 |
| Pentavitin (DSM) | SACCHARIDE ISOMERATE in aqueous solution at 50% MA | 0.5 |

1. A veterinary composition of the shampoo type, comprising an active ingredient obtained from Ophiopogon japonicus, and at least one amphoteric surfactant selected from the betaines.

2. The veterinary composition according to claim 1, comprising at least 0.1 wt.% active ingredient obtained from Ophiopogon japonicus, preferably between 0.1 and 1 wt.% with respect to the total weight of the composition, and more particularly between 0.1 and 0.6 wt.% with respect to the total weight of the composition.

3. The veterinary composition according to claim 1, said at least one amphoteric surfactant of the betaine type being selected from the alkyl(C8-C20)amidoalkyl(C1-C6)betaines, the alkyl(C8-C20)amidoalkyl(C1-C6)sulfobetaines, the alkyl(C8-C20)amidoalkyl(C1-C6)hydroxysulfobetaines.

4. The veterinary composition according to claim 1, comprising at least 3 wt.% amphoteric surfactants of the betaine type, preferably between 4 and 15 wt.% with respect to the total weight of the composition, and more particularly between 4 and 12 wt.% with respect to the total weight of the composition.

5. The veterinary composition according to claim 1, containing, in addition to said at least one amphoteric surfactant selected from the betaines, at least one anionic surfactant, at least one additional amphoteric surfactant, at least one nonionic surfactant, or a mixture thereof.

6. The veterinary composition according to claim 5, wherein the anionic surfactants are selected from disodium laureth sulfosuccinate, sodium lauroyl methyl isethionate, sodium methyl cocoyl taurate, or a mixture thereof.

7. The veterinary composition according to claim 5, wherein the additional amphoteric surfactant agent(s) in addition to those of the betaine type is (or are) selected from sodium cocoamphoacetate, sodium cocoyl glutamate, and a mixture thereof.

8. The veterinary composition according to any of claim 5, wherein the additional amphoteric surfactant(s) in addition to those of the betaine type are present in amounts of from 0.1 to 10 wt.%, preferably from 1 to 5 wt.%, and more particularly from 2 to 4 wt.%, and at best from 2 to 3 wt.%, with respect to the total weight of the composition.

9. The veterinary composition according to claim 5, wherein the composition comprises at least one nonionic surfactant selected from the alkyl (poly)glucosides, and in particular decyl glucoside, lauryl glucoside, cocoyl glucoside, capryl/caprylyl polyglucoside, or a mixture thereof.

10. The veterinary composition according to claim 5, wherein the nonionic surfactant(s) is (or are) present in the composition in amounts of from 0.1 to 15 wt.%, preferably from 1 to 10 wt.%, and more particularly from 3 to 8 wt.%, and at best from 5 to 7 wt.%, with respect to the total weight of the composition.

11. Veterinary composition according to any of claims 1 to 10, wherein the composition further comprises cationic galactomannan gums.

12. A method for cleaning and/or washing the skin and/or the coat of a non-human mammal, and in particular a dog or a cat, by applying the composition according to claim 1 to the skin and/or the coat of said mammal.

13. The method according to claim 12, characterized in that it comprises an application to the non-human mammal, optionally after having dampened all or some of the non-human mammal with water, and in that the foam generated, optionally by massage or friction using the hands, is subsequently removed, after an optional dwell time, by rinsing with water.

14. The method for reducing and/or preventing the formation and/or the adhesion of biofilms of pathogenic bacteria, or for reducing the presence of pathogenic bacteria on the skin or the coat, in a non-human mammal, in particular a dog or cat, by using the veterinary composition according to claim 1.

15. The method according to claim 14, characterized in that the pathogenic bacteria are selected from Staphylococcus aureus and/or Staphylococcus pseudintermedius.
